(19) United States
(12) Patent Application Publication
(10) Pub. No.: US 2021/0082548 A1
(43) Pub. Date: Mar. 18, 2021

(54) HEALTH PLATFORM

(71) Applicant: Bloxton Investment Group, LLC, Havertown, PA (US)

(72) Inventors: Michael Bloxton, Havertown, PA (US); Niki Faldemolaei, La Jolla (US)

(73) Assignee: Bloxton Investment Group, LLC, Havertown, PA (US)

(21) Appl. No.: 17/024,238

(22) Filed: Sep. 17, 2020

Related U.S. Application Data
(60) Provisional application No. 62/901,277, filed on Sep. 17, 2019.

(51) Int. Cl. G16H 10/60; G16H 80/00; G16H 50/30; G16H 20/60; G16H 20/30; G16H 20/70; G06Q 20/06; A61B 5/00

(52) U.S. Cl. CPC G16H 10/60; G16H 80/00; G16H 50/30; G16H 20/60; A61B 5/6801; G16H 20/70; G06Q 20/065; A61B 5/746; G16H 20/30

(57) ABSTRACT

A computer-implemented healthcare management platform includes a device-agnostic data collection engine that collects healthcare data from users and a cryptocurrency engine that allows the users to share their healthcare data with others in exchange for tokens and/or health insights.

### BACKGROUND

1. The Problem

1.1 Healthcare Prioritized Over Healthy Living

One of the greatest shortcomings of the payer provider healthcare system is its lack of engagement with healthy individuals. Healthcare systems are built to provide patients with care rather than preventive treatment, leading to an industry plagued by reaction. Due to these standards, it is estimated that 50% of the US population may be diagnosed with a preventable chronic illness in their lifetimes.

Today, if a user wants to seek out preventive insights, they must see their doctor, perform disease screening, manually identify risk factors, and discuss tips for a healthy and balanced lifestyle. This process is slow, expensive, and unconventional.

1.2 Siloed Health Data Streams

Healthy individuals who desire technology-driven health insights are currently at the whims of their digital health service providers. These providers each have control over a certain subset of health data (sleep, nutrition, exercise), which they use to provide users with restricted insights.

The preventive health industry recognizes that the future of healthcare lies in providing holistic health solutions, yet service providers are still strongly against pooling information. This is largely the result of a fear of competition, a lack of financial incentive, and an underappreciation of the power of the crowd.

At this crossroads, today's preventive healthcare industry can be likened to the narrative of the early HIV research era. During this decade-long period, researchers from around the world worked independently without discovering any significant solutions, until the University of Washington's Game Science department led an intervention.

Understanding that collaboration was in the best interest of the community, the department created an online puzzle game titled FoldIt that pooled the data sets of individual research groups' onto one platform and allowed any individual to try their luck at a solution.

To much surprise, the game attracted over 200,000 active users, resulting in a solution to a decade-old problem in the span of 10 days. By opening access to data and harnessing the power of the community these researchers were able to create solutions that were more effective, affordable, and scalable than any prior offerings.

The system herein seeks to provide the same crowdsourcing effect to the entire preventive health industry; allowing users to work together to collectively revolutionize the preventive healthcare system.

1.3 Inaccessibility of Healthy Data

Access to data from healthy people is one of the greatest challenges currently facing health researchers. Many of the data sets available to researchers are currently confined to data collected from unhealthy individuals, leading to a lack of optimized preventive health solutions.

Because of these challenges, better data collection and analytics tools are currently the top investment priority for healthcare organizations. “Healthy” data may provide new insights to researchers and create a baseline for new treatments and studies, as understanding why people are healthy can be just as vital as understanding why they are sick. This is the idea of studying the healthy end of the health bell curve instead of the middle—the extreme cases may hold the secret to better health.

Currently, companies are spending millions attempting to solve the healthy data access barrier, creating hundreds of data quality, integration, aggregation, extraction, and management tools. These tools allow healthy data to be pulled from select individuals but they fail to capture more than a small fraction of the total information provided by the greater healthy community.

As the preventive health space continues to expand beyond the current $125B market value and becomes a more integral part of the $7.4T healthcare industry, the need for better infrastructure is becoming more acute.

### BRIEF SUMMARY OF THE INVENTION

2. Summary of the Health Platform

The Health Platform described herein is a disruptive technology health platform that provides consumers with preventive healthcare solutions. With over 45% of the US population being affected by preventable chronic diseases alone, the Health Platform uses the technology herein to tap into a tremendous potential that may help hundreds of millions on their healthcare journeys.

The Health Platform combines an array of disruptive technologies to allow users to collect, control, and share their health data. This data can be shared with health researchers and developers to create research studies and preventive applications; leading to finer, cheaper, personally-tailored preventive insights for all.

Interactions on the Health Platform may be governed through the use of an Eternity cryptocurrency, which rewards people for interacting with and asking questions of their health data.

### BRIEF DESCRIPTION OF THE DRAWINGS

FIG. 1 shows systems and methods embodied in non-transitory computer readable medium configured to perform processing steps.

FIG. 1A shows a network diagram of different components of the system.

FIG. 1B certain hardware components for use with the system.

FIG. 2, a Comparison of Old and New Platform Paradigm, depicts and compares current inefficient practice with the present invention's frictionless process.

FIG. 3 shows a Sensor Properties Collected on the Health Platform and illustrates the present invention's process of collecting and aggregating health data from multiple sources.

FIG. 4 shows a Fitbit Heart Rate Overlayed Over MiBand HR Normalized Fitbit and MiBand Heart Rate and shows a comparison of unnormalized data with the present invention's normalized data.

FIG. 5 shows Peer to Peer Preventive Insights and portrays the value exchange between users and researchers.

FIG. 6 shows Screenshots that show typical information as might be displayed on mobile phones.

FIG. 7 show Revenue Split and depicts the reward distribution process.

FIG. 8 show The Health Platform Model and illustrates the transactional relationships between platform participants.

FIG. 9 show Common Token Uses and presents examples of participant actions that create value.

FIG. 10 show a Leaderboard Jackpot Scheme that shows how jackpot distributions are determined.

FIG. 11 show a Sample Scoring Schema and illustrates the present invention's token-staking method.

FIG. 12 show Sample Reward Redemption and shows items that might be purchased with tokens.

FIG. 13 show a Quadratic Scheme and explains how governance votes are allocated.

FIG. 14 show Quadratic Scheme in Practice and represents an example of vote allocation.

FIG. 15 show Data Prepping Scheme and characterizes a noise reduction process.

### DETAILED DESCRIPTION OF THE INVENTION

3. Solution: The Health Platform

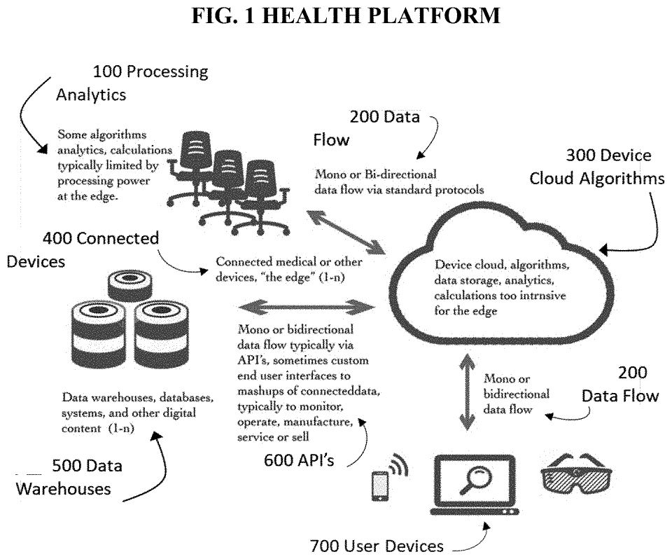

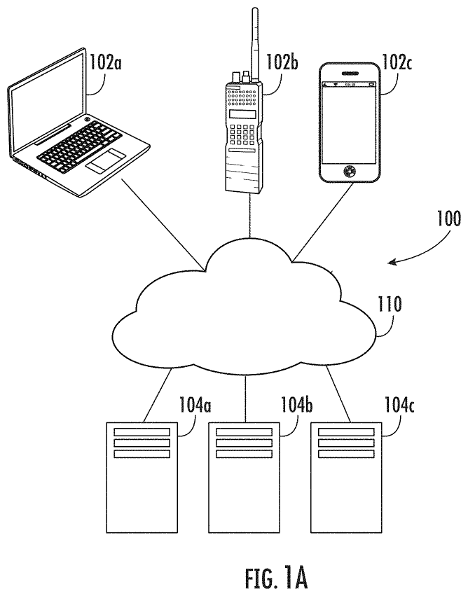

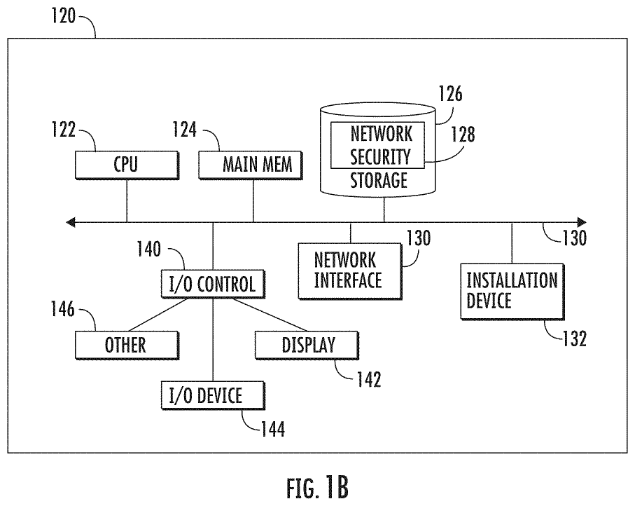

With respect to FIG. 1, which shows the Health Platform, Processing Analytics 100 are applied to Mono or bidirectional Data Flow 200 transmitted between Device Cloud Algorithms 300 and/or Connected Devices 400 and/or Data Warehouses 500 and processed between API's 600 and User Devices 700. Processing Analytics 100 are applied to Mono or bidirectional Data Flow 200 transmitted between Device Cloud Algorithms 300 and/or Connected Devices 400 and/or Data Warehouses 500 and processed between API's 600 and User Devices (IoT devices, smart phones, watches, computers, tablets, glasses, other wearable devices) 700.

The platform may provide foundational infrastructure for the preventive health industry. It may democratize access to usable health data, resolve health data silos, and provide a three-sided ecosystem for preventive healthcare.

The platform allows healthy people to collect data from their IoT devices (i.e wearables, diagnostics, or smart devices) and uses it to ask questions about their well-being.

Data is controlled by the end user, and can be shared with researchers and developers in exchange for cryptocurrency or cutting-edge, AI-driven health insights.

The core facilitator of network effects on the Health Platform is a virtual token called an Eternity token to differentiate it from other tokens. The Health Platform uses Eternity tokens to compensate users for sharing data, to fund new innovation, and to vote on upcoming platform decisions.

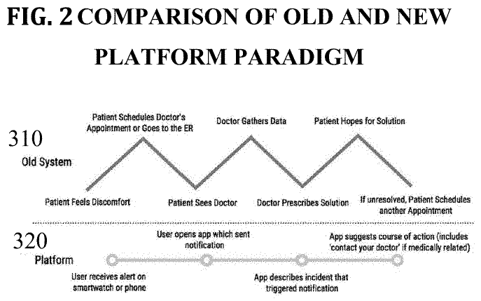

Referring to FIG. 2, in the old healthcare system 310, a patient goes back and forth with their doctor in several steps, which may result in a proscribed solution or referral. By comparison, the platform 320 may have an advice engine therein that provides alerts to devices to suggest courses of action for the user. In this way, the frictionless, low-cost accumulation and exchange of data is made possible through the innovation provided by an MS-based blockchain that allows for the creation of a preventive health industry built on collaboration and shared incentives.

3.1 The EOS Blockchain

The EOS blockchain has garnered significant investment due to its unique ability to transact value scalably and without central control.

EOS's distributed ledger lets users store and share their personal information without censorship, oversight, or tampering; providing individuals with a user-driven alternative to systems like banks or centrally-held data storage platforms. Given its scalability, versatility, smart contract capability, and peer to peer emphasis, EOS provides an optimal framework for the Health Platform.

All permissions on the Health Platform may be managed through smart contracts on the EOS blockchain. Users may have direct control over the EOS smart contract that grants others access to their anonymized and encrypted personal health data, and may be the only ones allowed to share their data with other researchers and developers.

The Health Platform may locate user data onto a separate, custom blockchain, or pursue transfer onto a chain capable of supporting privacy-preserving smart contracts. This may be done to bolster decentralization, enable added governance capabilities, and mitigate reliance on cloud services like AWS for the storage of user data.

In exchange for granting others access to their data, users may receive the Health Platform's native cryptocurrency, Eternity. Eternity may be an EOSIO token located on the EOS blockchain. Eternity may be transacted through the use of EOS smart contracts that may provide users with automated verification and payment processing.

EOS's blockchain may be advantageous over the known blockchains due to its scalability and near-zero transaction fees. This allows users to transact frequently and cost-effectively with minimal friction.

3.2 Data Collection

Data collection on the Health Platform may occur through integrated IoT devices, including wearables and diagnostics. The Health Platform may be used to store data from smart home devices (scales, cameras, mirrors etc), virtual assistants, motion detectors, and more. The platform may be initially integrated, for market access reasons, with full integration with an OURA RING, a wearable device for tracking sleep diagnostics, as well as other health wearables, with future integrations to come.

Integrated IoT devices may be initially connected through the PTC THINGWORX platform or similar IoT platform (as referred to generically herein), an industrial IoT platform capable of aggregating data and monitoring the performance of IoT devices. The IoT platform may allow users to connect all of their IoT devices and store their health data in one location. In other embodiments, data aggregation and performance monitoring may be transitioned directly onto an immutable, tamper-resistant blockchain, either through existing decentralized solutions like STREAMR or through the creation of the system's own blockchain.

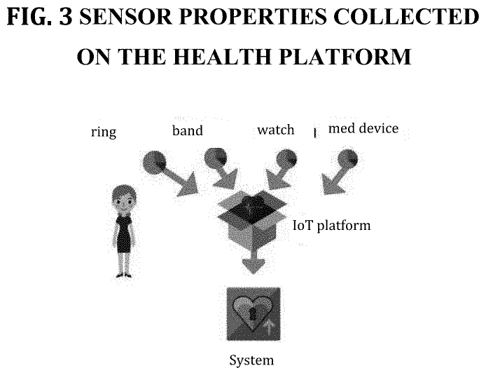

Referring to FIG. 3, once an IoT device has been integrated with IoT platform, raw device data, or as raw as device may allow, may flow seamlessly to user-controlled storage accounts. Health data may be temporarily stored using Google Cloud or AWS. Data may eventually be transitioned onto the EOS blockchain once inexpensive blockchain data storage is made possible.

3.3—Normalization

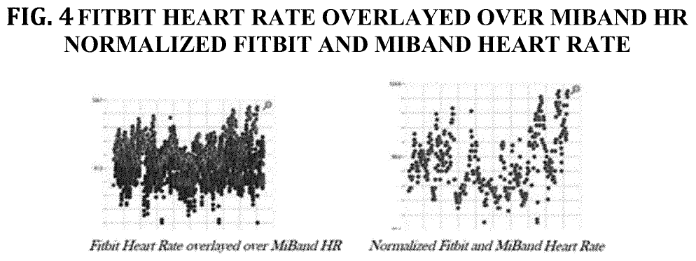

Referring to FIG. 4, data collected through the Health Platform may be normalized before being presented to end users. This allows data to be made readable and usable for preventive insights. Without data normalization insights remain siloed, and users are subject to the errors and variances of individual device sensors. This poses multiple potential issues that are common in the prior art.

For example, if a user wears their FITBIT incorrectly for even a single day, they are at risk of being diagnosed with a heart rate far from its true value. Using the Health Platform, the impact of outlier data may be mitigated, and more accurate results may be made apparent.

As a result of the Health Platform's innovation, sensor properties like heart rates and blood pressures may be able to be accurately measured no matter what device is connected.

This device-agnostic approach in which a device-agnostic data engine (the collection to a data warehouse 500 being shown in FIG. 1 at least) aggregates health data allows third party developers to build uniform applications on top of the Health Platform with a strict focus on the attribute data created by the individual sensors. This innovation in data collection and monitoring has the potential to yield groundbreaking results in preventive applications for heart disease, asthma, diabetes, depression, Alzheimer's, and more.

3.4—Privacy and Storage

User privacy is important in the modern age and a consideration for the Health Platform. Data passing through the IoT platform API may be anonymized and inaccessible to third parties, and may be compliant with the latest HIPAA, and GDPR standards. To access data, users may need to log on to their Health Platform account with their personal private key, which users store locally to ensure complete ownership over access privileges.

To share data with third parties, users can create a temporary access code through a trustless EOS smart contract. This grants data access to researchers for the duration of the research project they are pursuing, as well as to developers for one full year. This allows ample time for predictive application development.

Usernames, birthdates, and other sensitive information may be encrypted separately from health data, and may never be revealed to researchers or developers. Eventually, those looking for complete privacy preservation can purchase IoT devices directly through the Health Platform, which may be delivered with functionality that prevents manufacturers from accessing user health information without permission.

3.5—Research Studies

Both users and researchers may have the ability to use the Health Platform to coordinate the creation of unique preventive health studies. These studies reward individuals for being proactive about their health, providing metrics and other information on participants' well-being.

Research studies may be conducted using machine learning or artificial intelligence models to generate insights, which are then shared for the betterment of the platform community.

For example, a user on the Health Platform may notice that their baseline heart rate has been acting oddly over the last couple months. This user can then pose a question to the community asking what normal heart rates should look like, and enlist the assistance of researchers who may generate a “heart rate score” based on user information. This heart rate score can then be generalized and turned into a new data point for all Health Platform users (on top of their existing heart rate), which improves insights across the entire network.

Researchers on the Health Platform may initially be sought out from universities, research associations, and other reputable institutions, but may also be open to any individual capable of submitting strong solutions to user-generated questions. The inventors have already received interest from wearable manufacturers in creating and completing research studies, and sees this market as a prime fit for the offerings of the Health Platform.

To determine the most impactful/urgent insights, questions on the Health Platform may be prioritized based on the number of Eternity tokens that other users stake towards an answer. This is done to ensure that researchers are rewarded for their work and that top questions may stand out from others.

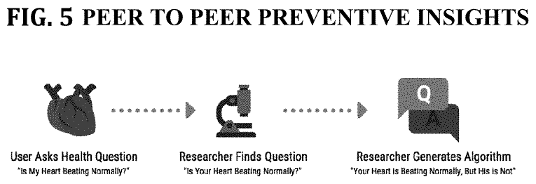

Referring to FIG. 5, researchers may additionally be able to ask and answer their own questions, offering payment for data from Health Platform users to create solutions. The first iteration of the Health Platform may feature a “pay-to-play” model, whereby research organizations can pay for user data and answer their own questions. The full launch of the two-sided research study marketplace may then be launched in future iterations.

3.6—Building Predictive Applications

Whenever a question is answered on the Health Platform, a new data point may be created that can be leveraged by third party application developers.

For example, the “heart rate score” from section 3.5 could be used to create applications that alert users whenever their heart rate is beginning to act irregularly.

The development of these predictive applications may be actively encouraged through the publishing of a Health SDK, which may allow developers to easily tap into insights from the Health Platform.

Developers can use this SDK to create monetizable applications that can be sold to any IoT device owners. Applications may be purchasable with Eternity tokens, and may split revenue from sales, downloads, ads, in-app items, and more between researchers, developers, and contributing users.

Applications developed using the Health Platform SDK may be listed exclusively on the Health Platform. This ensures that new application users may be presented with the chance to integrate their data onto the Health Platform and earn for sharing their data.

Preventive applications complete a health feedback loop, with data, research, and usable applications constantly being generated in one thriving ecosystem.

3.7—Design Alternatives

There are numerous ways in which platforms can provide users with control over their data. Completely decentralized approaches like storing all user data on a blockchain would accomplish this goal effectively, however would also result in significant expenses that render a platform designed for micro-payments entirely unusable. Additional privacy concerns with storing health data on a publicly verifiable ledger would also likely arise, as would the issue of preventing access to data from non-paying researchers.

4. Health Platform Application

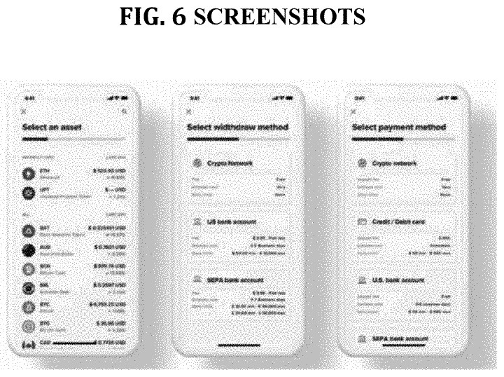

Referring to FIG. 6 the Health Platform Application provides open access to the Health Platform ecosystem. The application is the interface upon which users, researchers, and developers can collaborate and produce crowdsourced preventive health insights.

4.1—Creating an Account

Capturing value from the Health Platform is as easy as navigating to the device App Store and downloading the Health Platform application.

Stakeholders who download the Health Platform Application may receive a “registration bonus” of Eternity tokens, which may be collected in users' personal EOS wallets. Users can use the Health Platform without a connected wallet and may not collect Eternity tokens for their efforts.

After logging onto the platform, users can pair their wearable devices via bluetooth connection. This initiates IoT platform integration and data normalization. Users may then select the data they wish to have synced in real time, as well as how often they would like a sync to occur. The more often they sync, the more accurate the results.

Before participating in a study, users must fill out “one-time” information such as age and gender that may be required for all research studies. Information sharing may be rewarded with Eternity tokens.

4.2—Constructing Studies

The Health Platform Application assists both researchers and users with crafting well-designed studies. Whenever an individual has a question they would like to pose about data on the platform, the Health Platform App may guide them through the process of creating a standardized research study. This includes asking about the study's logistics, sample size, patient criteria, data points, and budget.

The Health Platform App may then dynamically suggest a realistic study design to engage the target audience, based upon a machine learning algorithm that has been trained from past successful studies. Researchers can abide by or reject suggestions; the application's goal is to strictly visualize the steps needed to create valuable research.

4.3—Joining Studies

Onboarded users can add themselves to an array of pre-designed user groups, which may be targeted by researchers for certain studies. These groups can be things like Athletes, Runners, Soccer Players, Bicyclists, and more. Users can also create their own custom groups to allow for coordination based on additional factors.

If uninterested in joining groups, users can browse the Health Platform app for research studies they would like to contribute to individually. Each project page shows how many other users are willing to contribute, how much users may be able to earn, and what the results of the study may produce.

If a project is of interest, users can commit to sharing their data upon launch as well as stake funds on the project to incentivize the initiation of research.

After staking funds, joining groups, and/or committing to share data with certain projects, users await the launch of research studies.

4.4—Launching Studies

After a sufficient number of users have agreed to commit their data to a study (as defined by the sample size), the project may move from the “Idea” stage to the “Open Studies” stage, where any researcher that is interested in the study can pitch themselves to take on the job. This is similar to a job application, where the hiring committee is the user base.

Once a researcher is approved to take on a study, they may gain access to live user data from the listed study commencement date until the listed study completion date. Funds that have been staked on the study may be held in escrow, with payments to the researcher or users occurring based on predefined milestones. Researchers may be expected to provide checkpoint reports indicating progress on the study in question. A small fee may be taken by the Health Platform for setting up this collaboration.

The end product of each research study may be the production of a “sound” machine learning model, as determined by a vote from the committed user base. Studies may be marked as completed once a researcher produces an algorithm that can generate a new data point for all participants in the study.

Upon completion, the researcher's name may be published alongside study results and metrics. Users may be able to vote on their researcher's performance and timeliness. This establishes a reputation system that holds researchers accountable for their work.

4.5—Creating Applications

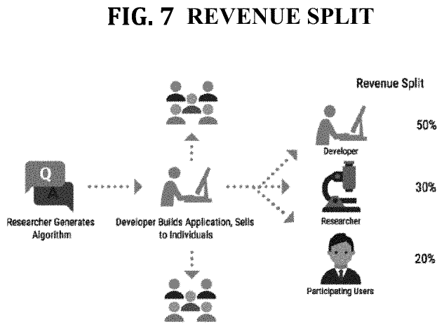

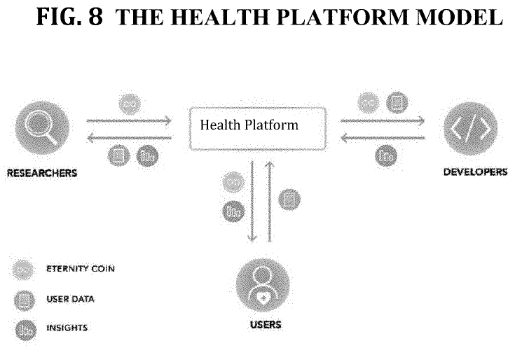

Developers can openly access the solutions provided by researcher's machine learning models through the Health Platform SDK. Referring to FIGS. 7 and 8, developers may be able to integrate researchers' machine learning models into user-friendly preventive applications, engaging in a revenue split with the researchers who created the model and the users who shared their data. FIG. 8 in particular shows the flow of Eternity coins as part of a cryptocurrency engine that allows the users to share their healthcare data with others in exchange for tokens and/or health insights.

For example, a research study could lead to the creation of a score that measures a user's sleep cycle. This score can be baked into an application that tracks sleep and incentivizes better sleep habits or as a single data point relating to athletic performance. Additional use cases of developer applications can be found in the Use Case section.

4.6—Health Platform Base Kit

Similar to the installation of the Apple operating system, the download of the Health Platform application may come with a full suite of subsidiary “PHealth” applications. These applications can be used to track items like the impact of a user's data on the lives of others, how much data a user has contributed, and what factors users should be aware of for their own health. Basic applications may include, but are not limited to:

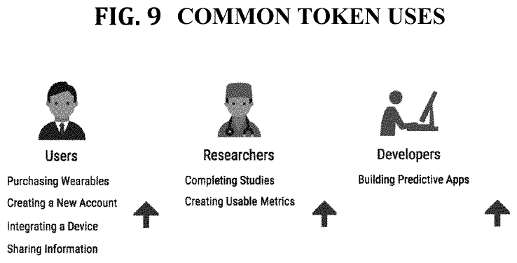

Help Data—An application that tracks the social impact of user data sharing. Referring to FIG. 9, users use this application to quantify how many people (users, researchers, developers) they have helped with their health data, as well as how many projects they have helped, days they have helped for, and cryptocurrency they have earned in exchange for their assistance.

Health Platform Leaderboard—A dynamic leaderboard that compares the data sharing of individuals to others in their region. Leaderboards feature a dynamic ranking of “Top Countries on the Health Platform”, which may rank countries by how much they are leading the charge in the creation of the preventive health economy. Users from the top country each month may receive a jackpot of Eternity tokens for their contributions to the platform.

Heart Rate Monitor—A preventive heart rate monitor that may notify users whenever their heart beat displays irregularities.

Stress Level Monitor—A stress level identification tool that monitors sensor levels, predicts stressful situations, and provides calming solutions.

5. Eternity Token

The Eternity Token may be an EOSIO standard cryptocurrency that provides governance privileges, rewards, and incentives to participants in the Health Platform ecosystem. The token allows for convenient and cost-effective access to Health Platform applications, insights, and supported IoT devices. Furthermore, the possession of Eternity provides holders with a suite of additional benefits.

5.1—Issuance

New users may receive Eternity tokens upon registration for the Health Platform. Tokens may be rewarded in fixed quantities by the Health Platform for sharing basic user information (age, sex, birthdate), and rewarded by researchers and developers for participating in studies and contributing to the creation of successful applications. Eternity tokens may also be rewarded by preventive health programs to incentivize healthy actions. For example, an anti-smoking institute could offer users a set amount of Eternity tokens each month in exchange for a sustained abstinence from smoking.

Eternity tokens may be rewarded to developers with each download of their preventive applications. Researchers may receive Eternity tokens for resolving studies requested by end users, as well as for contributing data points for predictive applications.

5.1.1—Leaderboard Jackpot

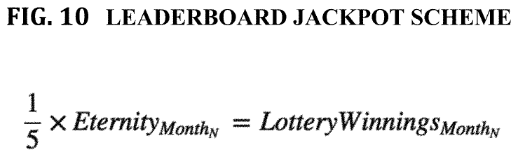

Each month, users from the top participating cities on the Health Platform may receive a jackpot of Eternity tokens for their contributions to the platform. Referring to FIG. 10, this contribution may be equal to ⅕ of all the Eternity tokens taken in via transaction fees and revenue share agreements. A live pot may be displayed on the “leaderboard” page, and may be distributed to the top 5,000 contributors from each city listed on the leaderboard.

5.2—Uses

5.2.1—Prioritizing Studies

Eternity tokens can be staked by users in order to promote or curate specific studies that are of high value to the Health Platform ecosystem.

For example, a user on the Health Platform may ask how far away they are from the optimal blood pressure level for their age and BMI. A personalized answer to this question could provide significant insights to the entire Health Platform community, and could thus be deemed a “high value question.”

In order to ensure that this question is prioritized over less impactful questions, users can ‘stake’ Eternity tokens on the question's resolution, meaning that they agree to pay the researcher who generates an answer to this question tokens in exchange for their work. This way researchers can be rewarded for improving preventive insights for the community, and the community can know that their important questions may be answered. Additionally, in order to encourage participation, users who stake tokens may receive a portion of the revenue generated by the new insights.

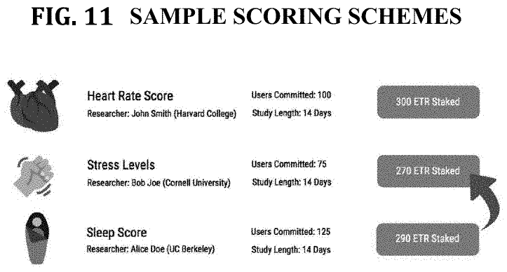

In this regard, staking tokens can be thought of as “curating” questions for researcher consumption. Referring to FIG. 11, a standard “highest-bidder” curation board may be generated on the Health Platform application, with the questions with the highest amount of Eternity (ETR) staked being placed at the top of the “Studies” list.

5.2.1—Purchasing IoT Devices

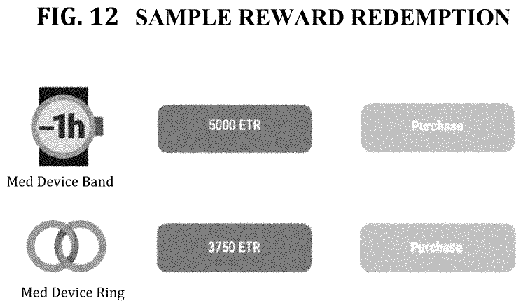

Eternity holders interested in growing their IoT device collection can spend Eternity to purchase new devices. Referring to FIG. 12, upon launch this may include the medical device ring, with future integrations to come. Upon reception, all devices can be immediately paired with the Health Platform, adding to the quantity of data that users can generate for research studies and the quantity of rewards users can earn for sharing their data.

5.2.3—Accessing User Data

Tokens can be utilized by researchers in order to access personalized user data for any study they seek to complete. Researchers simply post a study, list a compensation they are willing to pay in exchange for data, and pay that compensation at the completion or incrementally, such as hourly, until they have sufficient data for their study.

5.2.4—Governance over the Health Platform

In the spirit of open-source collaboration, all governance decisions on the Health Platform may be left into the hands of the Eternity token holder community. This includes the decision to build more built-in preventive applications, alter the reward distribution, and much more. This functionality may be instituted after the establishment of a sound Health Platform ecosystem, in order to ensure fair representation from users, researchers, and the developer community.

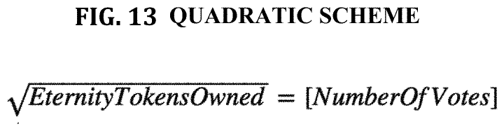

In order to prevent monopoly, governance may follow a quadratic scheme according to FIG. 13, with the number of votes a user receives corresponding to the square root of the number of tokens a user holds in their possession.

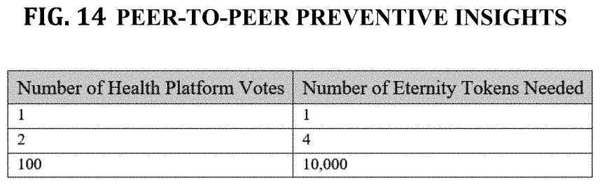

Referring to FIG. 14, this way it is difficult for an individual actor to attain 51% of the voting power required for a traditional network attack.

5.3—Health Platform Fund

In order to bootstrap initial adoption of the Health Platform, the Health Platform team may retain a sum of 100,000,000 Eternity tokens (10% of supply), used to provide grants to researchers and other stakeholders.

Grants are open to anybody in the Health Platform ecosystem with an idea that improves the Eternity platform for other users, whether it be an idea for a new research study, a new piece of platform functionality, or a new predictive application. Funds may be dispersed by a proposal review committee, consisting of Health Platform executives, advisors, and key stakeholders in the Health Platform ecosystem. Grant recipients may be expected to provide monthly progress reports on the development of their concepts in exchange for grant tokens.

The Health Platform Fund tokens may be issued into the economy at a rate of 20,000,000 tokens per year, in order to prevent the dilution of Eternity value due to reckless issuance. The inclusion of future governance capabilities may additionally enable the Health Platform network to set the inflation rate of Eternity tokens itself, free from the Health Platform team's oversight.

6. Use Cases

6.1—Stress Reduction

Stress is one of the world's greatest health influencers. Stress-generated conditions affect upwards of $1.3T USD a year in medical expenses, making it one of the core drivers of the health economy. Research shows that identifying stress levels and taking preventive measures increase productivity and lower health expenses, mitigating the likelihood of future exposure to heart disease, high blood pressure, obesity, cancer, and more.

Using smart watch data and simple machine learning models, the Health Platform platform may predict high-stress situations in advance of peak stress levels. This occurs through the measurement of heart rate and blood pressure from participating Health Platform users. The Health Platform platform and app may preemptively notify users of upcoming high stress environments; providing calming and soothing tips to keep stress levels under control.

6.2-21st Century Marathon

Today's marathon planning, setup, and configuration tools are outdated and un-optimized for runner performance. Designers of popular events like the New York City and Boston Marathon cite issues with crowd and runner safety, refreshment booth placement, and more leading to hundreds of thousands of dollars of cost and suboptimal marathon reviews.

By gathering data on runners' pace, heart rate, location, stamina levels, and fatigue points, brands and marathon organizers can use the Health Platform to re-engineer their course design for truly optimal performance. This enhances runner experience, race completion time, and safety of participants.

The Health Platform's functionality may include apps for beverage brands that predict where users may require refreshments, as well as apps for marathon creators looking to optimize their course design. The Health Platform may launch with stamina levels and fatigue points as two of the core data points available to applications developers, which can be used to create the first iteration of preventive Marathon applications.

6.3—High Performance Athletics

In today's digital age, winning in sports has become increasingly correlated with the strength of one's analytics department. Major sporting franchises are spending millions of dollars per year analyzing both up and coming prospects and first team players, in hopes of getting a step ahead of their competitors.

Two of the most pressing questions that each coach, GM, or manager seeks to answer with these analytics expenditures are “why are my top performers my top performers,” and “how can I tell why someone isn't performing at peak levels.”

The Health Platform may be the first platform to holistically be able to answer these questions. Through the normalization of data from performance wearables, microbiomes, smart devices, and biometric devices, researchers may possess adequate information to dissect a players performance and identify where shortcomings of athleticism occur. Whether it be due to a demonstrated lack of sleep the prior evening, a lack of water on the pitch, or an overall lack of conditioning, coaches and managers may be able to identify where player performance goes awry, and be able to generate solutions that prevent issues from arising again.

These solutions can have immediate impact for professional sporting franchises in both scouting and player development.

7. Technical Implementation

7.1—Data Collection and Storage

Data collection may occur through PTC's IoT platform, a centralized IoT system used for platform API's and data collection. Data measurement may initially be stored in a relational database (PostgreSQL), using IoT platform ValueStreams.

The Health Platform team may replace PostgreSQL with a streaming service and Kinesis analysis tool for Big Data applications. These tools are ideal for the Health Platform due to their scalability and performance relative to typical relational databases.

The Health Platform may additionally leverage the IoT platform Data Model to create dynamic views on sensors that can be applied (if needed) to devices automatically by the IoT platform system. In other words, whenever a user connects a device that is capable of providing new sensor types (i.e. glucose levels) that have not been provided previously, the IoT platform may automatically assign a corresponding name to this user device without user interaction with the IoT platform.

Each user registered on the Health Platform system (via a mobile application) may have a corresponding IoT platform account created, however, users may not have direct access to IoT platform.

7.2—Normalization of Collected Data

Normalization on the Health Platform entails the mapping of data from varying IoT devices to an underlying ground truth (a single sensor property). This may be accomplished using a series of statistical measures, all integrated onto the IoT platform.

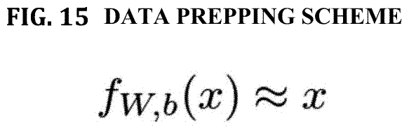

Referring to FIG. 15, once data is aggregated from a user's IoT device, data may be “prepped” by an autoencoder that is trained by the Predict team. This model may truncate excess data, remove outliers according to an acceptable standard deviation, and reduce overall noise using a combination of IQR and winsorization.

The Health Platform's autoencoder may first be trained on a ground truth representation. After training is completed, the encoder may use reconstruction error to normalize data.

7.3—Accessing User Data and Building Applications

3rd party application developers who would like to use data from the Health Platform application via the Predict SDK may have to be authenticated against IoT platform using a OOTB Application Key authorization mechanism, a IoT platform token that represents IoT platform user accounts.

Depending on permissions, the Health Platform team may be able to limit access to data that is stored in the IoT platform. For example, developers may be able to access a user's health sensor data, but may not be able to pull their name, birthdate, or email address. In the future, application key mechanisms may be used alongside Health Platform's on-chain blockchain system, to make data access directly resultant of smart contract transactions.

Access to a user's data currently occurs as follows:
A user gathers IoT data and aggregates it on the Health Platform (using IoT platform)
A smart contract is then created on the Health Platform blockchain
End-user data is tokenized, with the token being an application key that can be used to retrieve data from the IoT platform API
The token is stored in the user's smart contract
Researchers or developers decide to sign a user's smart contract to access data
Eternity from accessing party then transfers to end user
The user data token (application key) is simultaneously given to the accessing party
The accessing party now has access to user data

This process is a simplification of steps required to access user data, told through the lens of one user. In practice, accessing parties may ask for access to anonymized data from numerous users simultaneously. This process may look largely similar, with the introduction of additional transaction signatures acting as the core difference between interactions.

7.4—Sending Data to and from the Health Platform

The IoT platform Agent Webhook may be a middleware application that establishes websocket connectivity with the IoT platform. The Health Platform uses the IoT platform Agent Webhook to send data measurements to the Health Platform, create new devices in IoT platform, and control the variables that are assigned to IoT devices dynamically and automatically.

The IoT platform Agent Webhook additionally exposes the RESTful API, which can be used by the Health Platform's mobile application to push data from wearables and applications. In the future, any device that is smart enough to be onboarded with custom applications and internet connectivity without the need for a smartphone (or other device) may be using this RESTful API to push data to IoT platform.

7.5—Accessing the Health Platform

End users may be able to access the Health Platform via the Health Platform mobile application, available for download on devices. The Health Platform may be built on top of the Ionic CORDOVA framework, which allows the Health Platform developers to develop the application without touching device native code. The Health Platform may eventually create native applications on device platforms to allow for increased functionality and customization.

Mobile application users may be able to perform following actions:
Connect wearables to the system
Done using Bluetooth directly within the app, or via public cloud API
Review available studies and decide of participation
Download 3rd party applications developed on top of the Health Platform
Access the Leaderboard

The Health Platform mobile app may also interact directly with the Health Platform blockchain to complete data and Eternity token transactions. Upon registration, users may be set up with a supported EOS wallet, capable of amassing Eternity and other EOS tokens.

7.6—Encryption

The Health Platform places a strong emphasis on user security and data encryption, and may launch with full compliance with the latest HIPAA and GDPR standards.

In particular, upon the completion of all data normalization processes, each user's data may be bulk encrypted using the block cipher, and stored under each user's control. The TWOFISH block cipher was chosen for its balance between speed and security, as well as for its ability to use key whitening to reduce potential cryptanalysis and it additionally provides functionality for the stacking of further layers of encryption, meaning that the Health Platform developers could utilize it alongside other tools to provide users with further enhanced security.

8. System and Other Hardware Introduction

The system and method for the platform (including the cryptocurrency engine and other components) described may be implemented using system and hardware elements shown and described herein. For example, FIG. 1A shows an embodiment of a network 100a with one or more clients 102a, 102b, 102c that may be local machines, personal computers, mobile devices, servers, or tablets that communicate through one or more networks 110 with servers 104a, 104b, 104c. It should be appreciated that a client 102a-102c may serve as a client seeking access to resources provided by a server and/or as a server providing access to other clients.

The network 110 may be wired or wireless. If it is wired, the network may include coaxial cable, twisted pair lines, USB cabling, or optical lines. The wireless network may operate using BLUETOOTH, Wi-Fi, Worldwide Interoperability for Microwave Access (WiMAX), infrared, or satellite networks. The wireless links may also include any cellular network standards used to communicate among mobile devices including the many standards prepared by the International Telecommunication Union such as 3G, 4G, and LTE. Cellular network standards may include GSM, GPRS, LTE, WiMAX, and WiMAX-Advanced. Cellular network standards may use various channel communications such as FDMA, TDMA, CDMA, or SDMA. The various networks may be used individually or in an interconnected way and are thus depicted as shown in FIG. 1A as a cloud.

The network 110 may be located across many geographies and may have a topology organized as point-to-point, bus, star, ring, mesh, or tree. The network 110 may be an overlay network which is virtual and sits on top of one or more layers of other networks.

In most cases, every device on a network has a unique identifier. In the TCP/IP protocol, the unique identifier for a computer is an IP address. An IPv4 address uses 32 binary bits to create a single unique address on the network. An IPv4 address is expressed by four numbers separated by dots. Each number is the decimal (base-10) representation for an eight-digit binary (base-2) number, also called an octet. An IPv6 address uses 128 binary bits to create a single unique address on the network. An IPv6 address is expressed by eight groups of hexadecimal (base-16) numbers separated by colons.

An IP address can be either dynamic or static. A static address remains constant for a system unless modified by a user. Dynamic addresses are assigned by the Dynamic Host Configuration Protocol (DHCP), a service running on the network. DHCP typically runs on network hardware such as routers or dedicated DHCP servers.

Dynamic IP addresses are issued using a leasing system, meaning that the IP address is only active for a limited time. If the lease expires, the computer will automatically request a new lease. Sometimes, this means the computer will get a new IP address, too, especially if the computer was unplugged from the network between leases. This process is usually transparent to the user unless the computer warns about an IP address conflict on the network (two computers with the same IP address).

Another identifier for a device is the hostname. A hostname is a human-readable label assigned to a device and can be modified by a user. Hostname can be resolved to the IP address of the device. This makes hostname a more reliable device identifier in a network with dynamic IP addresses.

Information in the IP Address may be used to identify devices, geographies, and networks. The hostname may be used to identify devices.

A system may include multiple servers 104a-c stored in high-density rack systems. If the servers are part of a common network, they do not need to be physically near one another but instead may be connected by a wide-area network (WAN) connection or similar connection.

Management of group of networked servers may be de-centralized. For example, one or more servers 104a-c may include modules to support one or more management services for networked servers including management of dynamic data, such as techniques for handling failover, data replication, and increasing the networked server's performance.

The servers 104a-c may be file servers, application servers, web servers, proxy servers, network appliances, gateways, gateway servers, virtualization servers, deployment servers, SSL VPN servers, or firewalls.

When the network 110 is in a cloud environment, the cloud network 110 may be public, private, or hybrid. Public clouds may include public servers maintained by third parties. Public clouds may be connected to servers over a public network. Private clouds may include private servers that are physically maintained by clients. Private clouds may be connected to servers over a private network. Hybrid clouds may, as the name indicates, include both public and private networks.

The cloud network may include delivery using IaaS (Infrastructure-as-a-Service), PaaS (Platform-as-a-Service), SaaS (Software-as-a-Service) or Storage, Database, Information, Process, Application, Integration, Security, Management, Testing-as-a-service. IaaS may provide access to features, computers (virtual or on dedicated hardware), and data storage space. PaaS may include storage, networking, servers or virtualization, as well as additional resources such as, e.g., the operating system, middleware, or runtime resources. SaaS may be run and managed by the service provider and SaaS usually refers to end-user applications. A common example of a SaaS application is SALESFORCE or web-based email.

A client 102a-c may access IaaS, PaaS, or SaaS resources using preset standards and the clients 102a-c may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The clients 102a-c and servers 104a-c may be embodied in a computer, network device or appliance capable of communicating with a network and performing the actions herein. FIGS. 1A and 1B show block diagrams of a computing device 120 that may embody the client or server discussed herein. The device 120 may include a system bus 150 that connects the major components of a computer system, combining the functions of a data bus to carry information, an address bus to determine where it should be sent, and a control bus to determine its operation. The device includes a central processing unit 122, a main memory 124, and storage device 126. The device 120 may further include a network interface 130, an installation device 132 and an I/O control 140 connected to one or more display devices 142, I/O devices 144, or other devices 146 like mice and keyboards.

The storage device 126 may include an operating system, software, and a network user behavior module 128, in which may reside the network user behavior system and method described in more detail below.

The computing device 120 may include a memory port, a bridge, one or more input/output devices, and a cache memory in communication with the central processing unit.

The central processing unit 122 may be a logic circuitry such as a microprocessor that responds to and processes instructions fetched from the main memory 124. The CPU 122 may use instruction level parallelism, thread level parallelism, different levels of cache, and multi-core processors. A multi-core processor may include two or more processing units on a single computing component.

The main memory 124 may include one or more memory chips capable of storing data and allowing any storage location to be directly accessed by the CPU 122. The main memory unit 124 may be volatile and faster than storage memory 126. Main memory units 124 may be dynamic random-access memory (DRAM) or any variants, including static random-access memory (SRAM). The main memory 124 or the storage 126 may be non-volatile.

The CPU 122 may communicate directly with a cache memory via a secondary bus, sometimes referred to as a backside bus. In other embodiments, the CPU 122 may communicate with cache memory using the system bus 150. Cache memory typically has a faster response time than main memory 124 and is typically provided by SRAM or similar RAM memory.

Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, 3D printers, and VR headsets.

Additional I/O devices may have both input and output capabilities, including haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures.

In some embodiments, display devices 142 may be connected to the I/O controller 140. Display devices may include liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, VR or 3D displays.

The computing device 120 may include a network interface 130 to interface to the network 110 through a variety of connections including standard telephone lines LAN or WAN links (802.11, T1, T3, Gigabit Ethernet), broadband connections (ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols. The computing device 120 may communicate with other computing devices via any type and/or form of gateway or tunneling protocol such as Secure Socket Layer (SSL) or Transport Layer Security (TLS). The network interface 130 may include a built-in network adapter, network interface card, PCMCIA network card, EXPRESSCARD network card, card bus network adapter, wireless network adapter, USB network adapter, modem or any other device suitable for interfacing the computing device 120 to any type of network capable of communication and performing the operations described herein.

The computing device 120 may operate under the control of an operating system that controls scheduling of tasks and access to system resources. The computing device 120 may be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein.

The computer system 120 can be any workstation, telephone, desktop computer, laptop or notebook computer, netbook, tablet, server, handheld computer, mobile telephone, smartphone or other portable telecommunications device, media playing device, a gaming system, mobile computing device, or any other type and/or form of computing, telecommunications or media device that is capable of communication including the medical devices discussed above.

The status of one or more machines 102a-c, 104a-c may be monitored, generally, as part of network management. In one of these embodiments, the status of a machine may include an identification of load information (the number of processes on the machine, CPU and memory utilization), of port information (the number of available communication ports and the port addresses), session status (the duration and type of processes, and whether a process is active or idle), or as mentioned below. In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

While the invention has been described with reference to the embodiments above, and illustrated in FIG. 1, a person of ordinary skill in the art would understand that various changes or modifications may be made thereto without departing from the scope of the claims.

The descriptions and function of the various embodiments described above can be combined to provide further embodiments. Additional changes can be made to the systems and methods in the above description.

The following claims and terms herein should not be rendered to limit the systems and methods to the embodiments disclosed in the specification and the claims, but should be understood to include all processing systems that operate under the claims. Accordingly, the systems and methods are not limited by the specific examples, but instead the scope of the systems and methods is to be determined entirely by the claims.

While certain examples of the systems and methods are presented below in the claim forms, the inventors intend the various aspects of the systems and methods present in any number of claim forms. As an example, while only one aspect of the systems and methods are be recited as embodied in non-transitory computer readable medium, other aspects may likewise be embodied in non-transitory computer and machine-readable medium. Accordingly, the inventors reserve the right to add additional claims after filing the application to pursue such additional claim forms far evolving aspects of the systems and methods.

What is claimed is:

1. A computer-implemented healthcare management platform comprising:
a device-agnostic data collection engine that collects healthcare data from users; and
a cryptocurrency engine that allows the users to share their healthcare data with others in exchange for tokens and/or health insights.

2. The computer-implemented healthcare management platform of claim 1, wherein the users are granted the tokens in exchange for sharing their healthcare data following execution of a smart contract.

3. The computer-implemented healthcare management platform of claim 1, wherein the healthcare data provided by the users is provided by one or more of a group consisting of: wearable monitors, home monitors, virtual assistants, motion detectors, other IoT devices.

4. The computer-implemented healthcare management platform of claim 1, further comprising an advice engine that provides alerts to the user regarding changes and/or proactive steps to undertake for their health.

5. The computer-implemented healthcare management platform of claim 1, wherein the healthcare data is transitioned into a blockchain.

6. The computer-implemented healthcare management platform of claim 1, wherein the healthcare data is anonymized before sharing with the others.

7. The computer-implemented healthcare management platform of claim 1, wherein the token provides governance privileges, rewards, and incentives to users and others.

8. The computer-implemented healthcare management platform of claim 1, wherein tokens represent voting power.

9. The computer-implemented healthcare management platform of claim 8, wherein voting power follows quadratic scheme, with the number of votes a user receives corresponds to the square root of the number of tokens a user holds in their possession.
